(19) United States
(12) Patent Application Publication
(10) Pub. No.: US 2025/0352159 A1
(43) Pub. Date: Nov. 20, 2025

(54) METHOD OF QUANTIFYING AND SCORING LUNG MUCUS PLUGS

(71) Applicant: Vida Diagnostics, Inc., Coralville, IA (US)

(72) Inventors: Samuel W. Peterson, Topanga, CA (US); John D. Newell, JR., Port Townsend, WA (US); Benj Thomas, Eden Prairie, MN (US); Sandra Stapleton, Redmond, WA (US); Todd Johnson, Chanhassen, MN (US)

(21) Appl. No.: 19/210,304

(22) Filed: May 16, 2025

Related U.S. Application Data
(60) Provisional application No. 63/648,953, filed on May 17, 2024.

(51) Int. Cl. A61B 6/00; A61B 6/50; G06T 7/00; G06T 7/62; G16H 50/30

(52) U.S. Cl. CPC A61B 6/5217; A61B 6/50; G06T 7/0016; G06T 7/62; G16H 50/30; G06T 2207/10081; G06T 2207/30061

(57) ABSTRACT

A method of providing a lung airway mucus plug score from image data includes determining a weighted sum of the mucus plugs based on the airway generation at which the mucus plug occurs. A method of providing a lung airway mucus plug score from image data includes determining a total obstructed cross-sectional area of the mucus plugs. A method of providing a lung airway mucus plug score from image data includes determining a total count of obstructed airway branches that have one or more mucus plugs.

### CROSS-REFERENCE TO RELATED APPLICATIONS

This application claims priority to U.S. Provisional Patent Application No. 63/648,953, filed May 17, 2024, the contents of which are incorporated herein by reference in its entirety.

### TECHNICAL FIELD

This disclosure is generally directed to the use of medical imaging data to assess the extent of lung mucus plugs in the airways of patients. More particularly, this disclosure describes a method of using computed tomographic (CT) image data to provide a quantitative assessment or “severity score” of the presence of mucus plugs in the airways of a patient's lungs.

### BACKGROUND

Increased mucus in the airways of the lungs is associated with several different pulmonary diseases. Mucus can build up in the airways to the point of plugging the airway lumen and impairing air flow to the more distal smaller airways of the lungs. Various therapies for clearing mucus are available and under development, making it useful to be able to quantify mucus levels, for instance, before and after treatment. Airways that are plugged by mucus can be identified on x-ray computerized tomographic (CT) scans of the thorax. In some cases, it may be desirable to quantify the degree or extent of mucus plugs and/or to determine mucus plugging severity within a lung.

Thus, there exists a need for improved methods and systems for quantifying and/or scoring the extent of lung mucus plugging in a patient's lungs. Likewise, there exists a need for improved methods and systems for characterizing mucus plug burden in the lungs ranging from a local level (e.g., sub-lobe or finer) to a global level (e.g., whole lung).

### SUMMARY

Certain embodiments of this disclosure are described herein with reference to illustrative embodiments.

This disclosure describes methods of quantifying or scoring the extent of mucus plugging (the mucus plug burden) to thereby provide a standard and/or a criterion by which to characterize or categorize the severity of mucus plugging for a given patient or among patients. The characterization of airway mucus plug burden may be applied at varying levels ranging from local (e.g., sub-lobe or finer) to global (whole lung), according to various embodiments. The characterizations or measurements may be used to capture the number and distribution of mucus plugs relative to the overall anatomy (e.g., lungs, lobes, and airway tree).

Some embodiments of this disclosure include methods of providing a mucus plug score comprising a weighted sum of the mucus plugs, where each mucus plug's contribution to the score is based on the airway generation at which the mucus plug occurs (e.g., aggregated by whole lung, right/left, lobe, and/or sub-lobe). Additionally, in some embodiments, grouping/aggregating/parsing of the mucus plug scores (or of any related measures) may be performed according to airway generation level.

Some embodiments of this disclosure include methods of providing a mucus plug score based on the total obstructed cross-sectional area of the mucus plugs. For example, the mucus plug score may be a total cross-sectional area in some embodiments, or it may be a relative score that is normalized for example by a patient's total lung volume, or total cross-sectional area, or the cross-sectional area of segmental airway segments only (e.g., aggregated by whole lung, right/left, lobe, and/or sub-lobe). Additionally, in some cases, grouping/aggregating/parsing of the mucus plug scores (or of any of the related measures) may be performed according to airway generation level. For example, it may be desirable to report total obstructed cross-sectional area for mucus plugs occurring at each generation level.

Some embodiments of this disclosure include methods of providing a mucus plug score comprising a total plug mass of the mucus plugs, determined as the product of volume and density of the plugs, and summed for all the mucus plugs in a relevant portion of the lungs (e.g., aggregated by whole lung, right/left, lobe, and/or sub-lobe). Additionally, in some cases, grouping/aggregating/parsing of the total plug mass may be performed according to airway generation level. For example, it may be desirable to report total plug mass for mucus plugs occurring at each generation level.

Some embodiments of this disclosure include methods of providing a mucus plug score based on plug distribution. For example, the spatial density of the mucus plugs (e.g., number of plugs per cubic centimeter of lung) may be provided (e.g., aggregated by whole lung, right/left, lobe, and/or sub-lobe).

### BRIEF DESCRIPTION OF DRAWINGS

The following drawings are illustrative of particular embodiments of this disclosure and therefore do not limit the scope of this disclosure. The drawings are not necessarily to scale (unless so stated) and are intended to accompany the explanations in the following detailed description. Embodiments of this disclosure will hereinafter be described in conjunction with the appended drawings, wherein like numerals denote like elements.

FIG. 1 is a scan of a portion of a lung with a mucus plug in an airway;

FIG. 2 is a scan of a portion of a lung with the airway being open;

FIG. 3 is a flow diagram showing steps of a method for determining a mucus plug score according to some embodiments of this disclosure;

FIG. 4A is a schematic diagram showing an exemplary airway branch structure for right and left lungs of a patient according to some embodiments of this disclosure;

FIG. 4B is a schematic diagram showing an exemplary airway branch structure for right and left lungs of a patient according to some embodiments of this disclosure;

FIG. 4C is a schematic diagram showing an exemplary airway branch structure for right and left lungs of a patient according to some embodiments of this disclosure;

FIG. 5 is a series of exemplary formulas for calculating a mucus plug score according to some embodiments of this disclosure;

FIG. 6 is a schematic diagram showing an exemplary airway branch structure with mucus plug locations indicated to illustrate calculation of a mucus plug score according to some embodiments of this disclosure;

FIG. 7 is a flow diagram showing steps of a method for determining a mucus plug score according to some embodiments of this disclosure;

FIG. 8 is a schematic perspective diagram of an exemplary airway branch with a mucus plug to illustrate calculation of a mucus plug score according to some embodiments of this disclosure;

FIG. 9 is a flow diagram showing steps of an alternative method for determining a mucus plug score according to some embodiments of this disclosure;

FIG. 10 is a schematic diagram showing an exemplary airway branch structure with mucus plug locations indicated to illustrate calculation of a mucus plug score according to some embodiments of this disclosure; and

FIG. 11 is a flow diagram showing steps of a method for determining a mucus plug score according to some embodiments of this disclosure.

### DETAILED DESCRIPTION

This disclosure describes one or more methods of quantifying or scoring the extent of mucus plugging (the mucus plug burden) to provide a standard and/or a criterion by which to characterize or categorize the severity of mucus plugging for a given patient or among patients. The methods described herein may improve the consistency and therefore the clinical relevance of such assessments and thereby enable better clinical decision making.

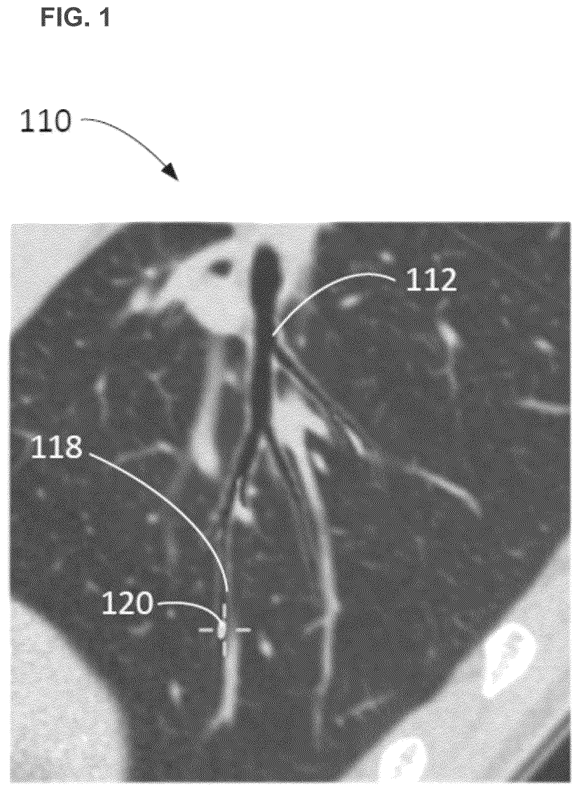

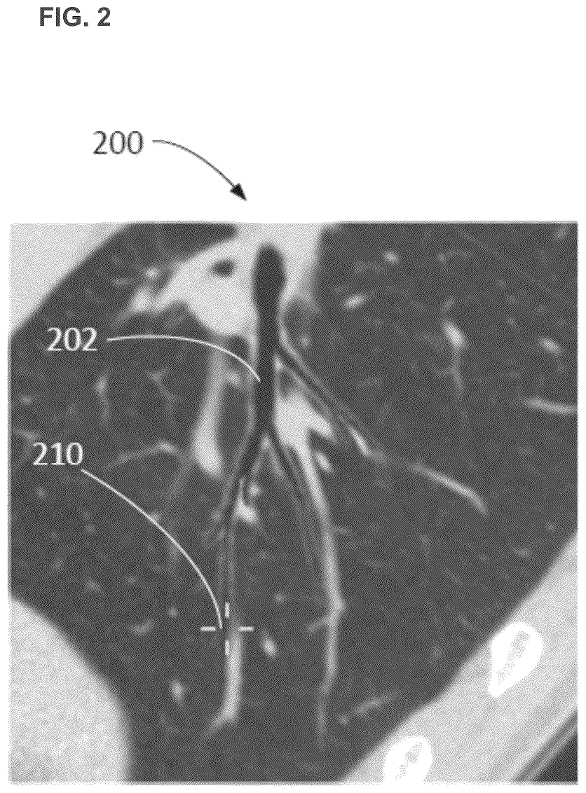

FIG. 1 is a scanned image 110 (e.g., from a CT scan) of a portion of a lung having a sub tree 118 of an airway 112 being blocked with a mucus plug 120. FIG. 2 is a scanned image 200 of a portion of a lung similar to the one shown in FIG. 1 with the sub tree 210 of the airway being open (e.g., no mucus plug blocking the airway).

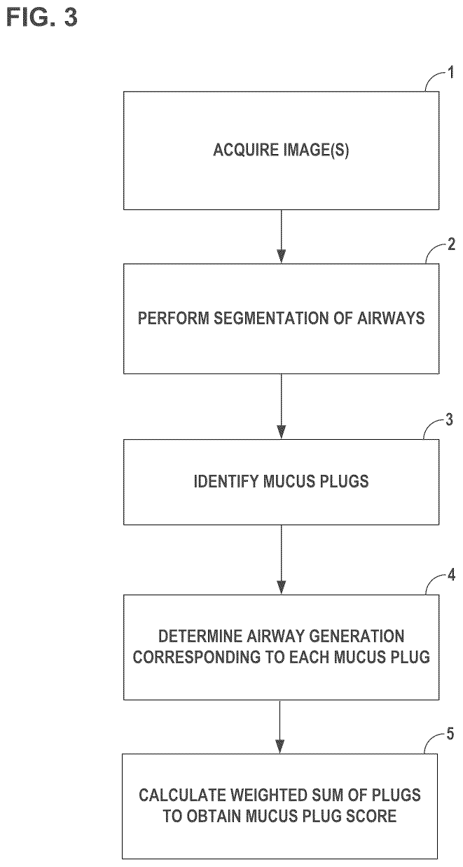

FIG. 3 is a flow diagram showing exemplary steps of a method of assessing mucus plugs in a lung of a patient according to certain embodiments of this disclosure. At step 1, images of the lung (or lungs, or a portion of a lung) are acquired. For example, radiological images or imaging data of a patient's lungs are transmitted to a pulmonary imaging system. The radiological images (e.g., volumetric radiological images) or imaging data may include CT scanned images or MRI scans, for example, from which a series of two-dimensional planar images can be produced in multiple planes, for example. Each image in the series of the multi-dimensional volumetric images provided by CT and MRI scans, for example, is a two-dimensional planar image that depicts the tissue present in a single plane or slice. These images are typically acquired in three orthogonal planes, which are referred to as the three orthogonal views and are typically identified as being axial, coronal and sagittal views.

At step 2, segmentation of the airways may be performed. It should be noted that airway segmentation may not need to be performed in all embodiments described herein. For example, airway segmentation might be performed only to the extent necessary to assess the generation level of the mucus plug locations. In some cases, airway segmentation may be performed to help delineate the segments (e.g., sub-lobes). In certain other embodiments, airway segmentation may be performed to help calculate a normalized value of a mucus plug score, for example. However, even in such cases, it may not be necessary to segment the entire airway tree. Continuing with step 2 of FIG. 3, the lungs, airways, and/or blood vessels may be segmented using the volumetric image data acquired in step 1. In some embodiments, a method may include processing the received volumetric pulmonary scan data to identify one or more anatomical structures within the volumetric pulmonary scan data. The methods of performing lung, airway and vessel segmentation from the volumetric images or imaging data may be those employed by the Pulmonary Workstation of VIDA Diagnostics, Inc. (Coralville, Iowa) and as described in the following references, each of which is incorporated herein by reference in relevant part: U.S. Pub. 2007/0092864, entitled, “Treatment Planning Methods, Devices and Systems”; U.S. Pub. 2006/0030958, entitled, “Methods and Devices for Labeling and/or Matching”; U.S. Pub. 2023/0363730, entitled, “Airway Mucus Visualization”; Tschirren et al., “Intrathoracic airway trees: segmentation and airway morphology analysis from low-dose CT scans,” IEEE Trans Med Imaging. 2005 December; 24 (12): 1529-39; Tschirren et al., “Matching and anatomical labeling of human airway tree,” IEEE Trans Med Imaging. 2005 December; 24 (12): 1540-7; Tschirren, Juerg, “Segmentation, Anatomical Labeling, Branchpoint Matching, and Quantitative Analysis of Human Airway Trees in Volumetric CT Images,” Ph.D. Thesis, The University of Iowa, 2003; Tschirren, Juerg, Segmentation, Anatomical Labeling, Branchpoint Matching, and Quantitative Analysis of Human Airway Trees in Volumetric CT Images, Slides from Ph.D. defense, The University of Iowa, 2003; and Li, Kang, “Efficient Optimal Net Surface Detection for Image Segmentation—From Theory to Practice,” M.Sc. Thesis, The University of Iowa, 2003. Segmentation of the lungs, airways, and vessels results in identification of the lungs, airways, and vessels as distinct from the surrounding tissues and of separation of the lungs, airways, and vessels into smaller distinct portions which may be individually identified in accordance with standard pulmonary anatomy.

Lobar segmentation may optionally be performed. The segmentation of the lungs, airways, and vessels obtained in step 2 can be used to identify and delineate the lobes, again by applying standard pulmonary anatomy. For example, using the identified segments of the airway and/or vessel trees obtained in step 2, the lobes may be segmented and identified by extracting the portions of the airway tree corresponding to particular lobes based on known airway tree structures and connectivity information. The extracted lobar airway tree portions may be further divided into portions corresponding to sub-lobes, again based on known airway and/or vessel tree structure and connectivity information. In this way, the portions of the volumetric images corresponding to lobes and/or sub-lobes can be identified.

With continued reference to FIG. 3, step 3 involves identifying mucus plugs in the segmented images obtained from steps 1 and 2. A mucus plug may be formed by the accumulation of mucus in the lungs that can reduce, obstruct, or occlude airflow in the airways of the lungs. Mucus plugs occluding multiple bronchi may increase the risk of pneumonia and COPD exacerbations, for example. Criteria for identifying and/or confirming the existence of a mucus plug may include, for example, the presence of an open airway distal to the mucus plug location, among other things. Examples of some methods for identifying mucus plugs are provided, for example, in U.S. Publication 2023/0363730 A1, entitled, “Airway Mucus Visualization,” the contents of which are hereby incorporated by reference in relevant part. The identification of mucus plugs may be performed automatically (e.g., via a neural network with deep learning trained to identify mucus plug candidates), manually (e.g., by experts or non-experts), or using a combination of both. In some examples of automated identification, forms of artificial intelligence other than neural networks may be used to detect, identify, and/or confirm mucus plug candidates. Additionally or alternatively, in some examples, non-artificial intelligence computerized analysis may be used to detect, identify, and/or confirm mucus plug candidates. Additionally or alternatively, in some examples, the location of a patient's previously identified mucus plugs (e.g., from a prior data set) can be used as presumptive locations of their current mucus plugs. In some examples of manual identification, instead of an expert manually identifying mucus plug candidates, non-experts can be trained to identify possible mucus plug candidates, which can then be reviewed by an expert later. By having non-experts identify mucus plug candidates, the time and resources required by the expert are reduced such that the expert need only review mucus plug candidates. In some examples, non-experts can be another step in the process whereby the non-experts review mucus plug candidates identified by a neural network before providing their results to an expert for final review.

In step 4, for each mucus plug identified in step 3, the “airway generation” may be determined or assigned corresponding to the location of each mucus plug in the airways of the lungs. The “airway generation” is a measure, or an indication, or a number, generally indicating how deep into the lung airway branch structure a particular location is. A number of different airway generation numbering schemes may be possible. Some exemplary airway generation numbering schemes are described in more detail hereinafter as possible ways of classifying the locations of mucus plugs in accordance with various embodiments of this disclosure.

At step 5, a mucus plug score (or mucus plug severity score) is determined and/or calculated. The mucus plug score may be a weighted sum of the mucus plugs, where the weighting factor (or coefficient) assigned to a given mucus plug is based on the airway generation in which the mucus plug is located. Generally, the airway branches that are in earlier generations (e.g., airway branches that are closer to the trachea, for example) will be weighted more heavily (have a higher weighting factor) than airway branches in later generations. For example, a mucus plug located in a 3^{rd} generation airway branch (e.g., three branches down from the trachea) might be assigned a weighting factor that is higher than a mucus plug located in a 7^{th} generation airway branch (e.g., seven branches down from the trachea). An example calculation may illustrate the computation of a mucus plug score according to some embodiments of this disclosure. In an exemplary scan, 4 mucus plugs are identified as follows:
1 mucus plug located in a 3^{rd} generation airway branch; and
3 mucus plugs located in a 7^{th} generation airway branch.

The Mucus Plug Score (“MPS”) may be determined as:

$$\begin{matrix}MPS = ( 1 ) {xCF}_{3} + ( 1 ) x C {F}_{7} + ( 1 ) x C {F}_{7} + ( 1 ) x C {F}_{7} \\ MPS = ( 1 ) x ( 0.8 ) + ( 1 ) x ( 0.3 ) + ( 1 ) x ( 0.3 ) + ( 1 ) x ( 0.3 ) = 1.7\end{matrix}$$

It should be noted that the generation-based coefficients (or weighting factors) used in the example above are by way of example only and are not intended to be limiting in any way. The results would be useful for comparison purposes once a suitable set of weighting factors is chosen or evolves from usage, for example.

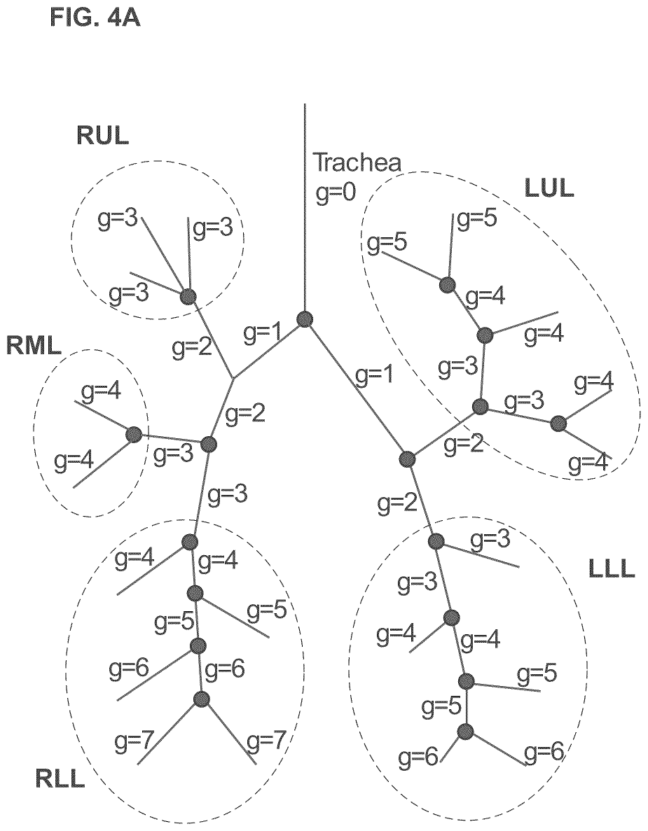

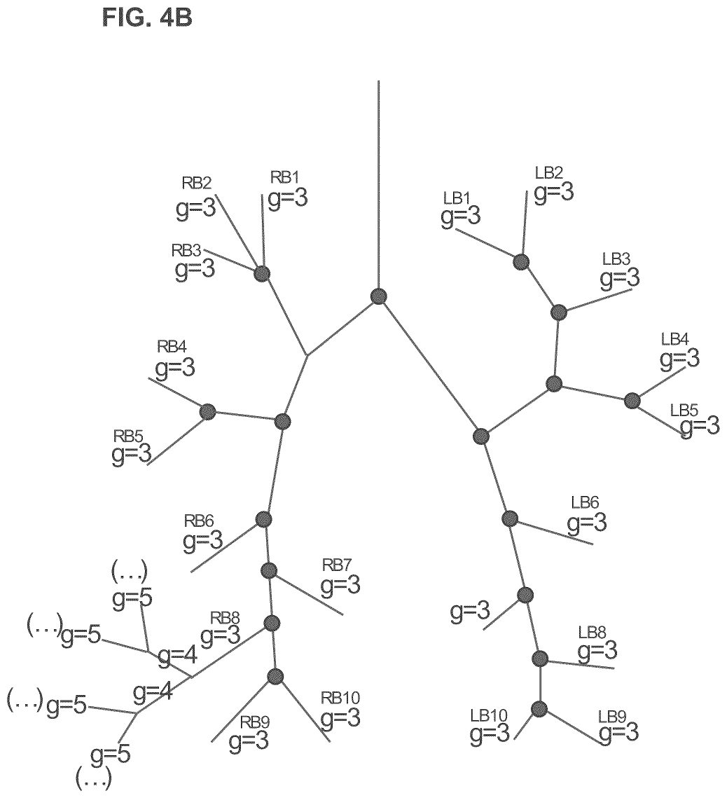

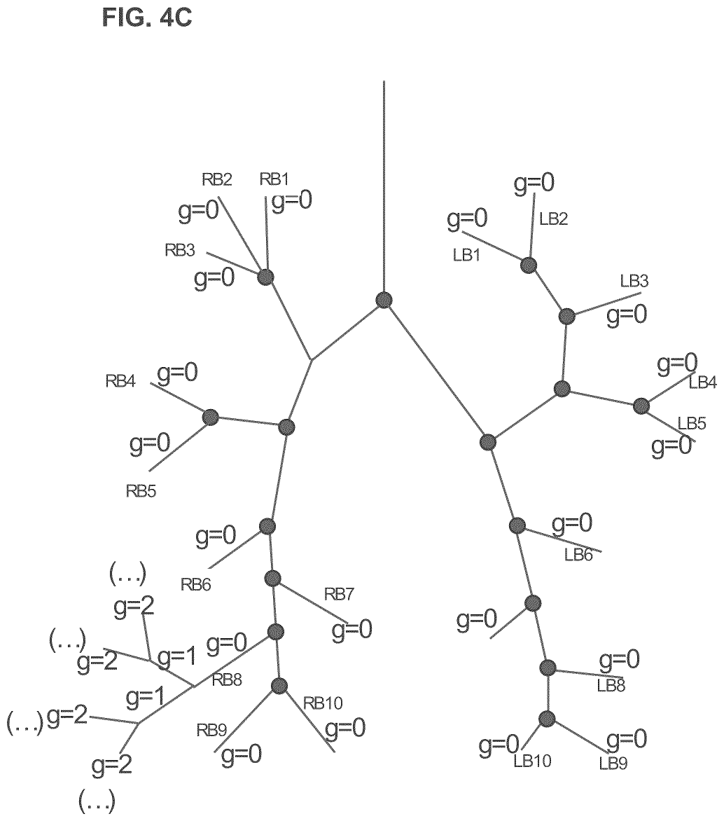

Referring back to step 4 of the method shown in FIG. 3, one example of an airway generation numbering scheme is provided in FIG. 4A. In the example shown in FIG. 4A, an “absolute/global” airway generation scheme is depicted. For example, starting at the top/center, the trachea is indicated as being in generation “0” (“zero”) in this scheme, and each successive “branch” of the airway tree results in a subsequent generation number. The trachea first branches into the right and left main bronchi at the tracheal carina, the right and left main bronchi indicated as being in generation “1” in this generation numbering scheme. At the next bifurcation or branch (e.g., towards the right upper lobe, “RUL,” and towards the left upper lobe, “LUL”), a pair of airway segments is indicated as being in generation “2.” Within RUL, the three branches (which may be referred to as the RB1, RB2, and RB3 segmental airways) are indicated as being in generation “3.” Using this scheme, the numbering process continues with a successive generation number corresponding to each successive bifurcation in the airway branch. For example, the segmental airway RB10 (of the RLL) would be indicated as being in generation “7” in this generational numbering scheme.

Referring back to step 5 of the method shown in FIG. 3, the weighting factor for a given mucus plug may be determined by the generation. Thus, in one possible embodiment, all mucus plugs in a 3^{rd} generation airway branch would be assigned the same weighting factor (coefficient), all mucus plugs in a 4^{th} generation airway branch would be assigned the same weighting factor (coefficient), all mucus plugs in a 5^{th} generation airway branch would be assigned the same weighting factor (coefficient), etc., and the weighting factor for the 3^{rd} generation would be greater than the weighting factor for the 4^{th} generation, and the weighting factor for the 4^{th} generation would be greater than the weighting factor for the 5^{th} generation, etc.

It should be noted that, in some cases, the coefficients for successive generations may not necessarily always decrease with higher generation numbers. For example, segmental airway branches LB10 and RB10 (which have fairly high global generation numbers) are typically wider than certain other airway segments, such as RB1, RB2, and RB3, which have relatively low global generation numbers. These kinds of discrepancies and non-uniformity of the airway tree makes the scheme of weighting plugs by coefficient somewhat attractive. For example, the coefficients for LB10 and RB10 could be chosen to more accurately reflect the relative sizes of the airways (e.g., without being tied solely to the generational number assigned).

An alternative example of an airway generation numbering scheme is provided in FIG. 4B. In the example shown in FIG. 4B, a “segmental” airway generation scheme is depicted. In some cases, it may make sense to treat the occurrence of mucus plugs in the segmental airway branches as being of equal weight. For example, the approximately 18-20 segmental airway branches (e.g., RB1 through RB10, and LB1 through BL10, with some variation in whether to include LB7 and/or whether to treat LB1/LB2 together) are all indicated in FIG. 4B as being in generation “3” for the purposes of this generational schema. As shown, there may be airway branches downstream of the segmental airways, such as those indicated beyond RB8 in FIG. 4B. Successive branches beyond the segmental airways are treated as further generations at each successive bifurcation, with 4^{th} and 5^{th} generation segments indicated downstream of RB8 in FIG. 4B.

FIG. 4C is a schematic diagram showing a variation of the exemplary airway branch structure of FIG. 4B according to some embodiments of this disclosure. In FIG. 4C, for example, each of the segmental airway branches is indicated as being in generation “0” for the purposes of this generational schema. Other exemplary numbering schemes are contemplated as may be deemed suitable for use by those of ordinary skill in the art.

It is worth noting that, in some cases, the presence of at least one mucus plug in a given airway branch or segment is sufficient to “turn on” the weighting (based on airway generation) and have it be included in the computation of a Mucus Plug Score, for example. In certain embodiments, the presence of more than one mucus plug in a given airway segment may not contribute further or add to the computed mucus plug score. In such an embodiment, for example, a single mucus plug in a given segmental airway (e.g., RB1) might contribute the same amount to the total calculated Mucus Plug Score as would having two, three or four (or more) mucus plugs located in RB1.

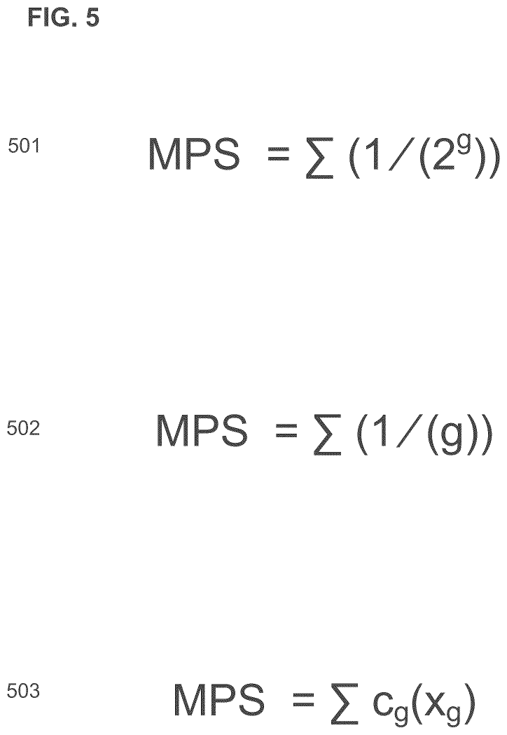

FIG. 5 shows a number of exemplary equations or formulas for computing a mucus plug score in accordance with embodiments of this disclosure. For example, equation 501 describes a computation of a mucus plug score in which each mucus plug identified in the scanned image contributes to the mucus plug score an amount inversely related to the airway generation as follows:

$$mucus plug score = \sum ( 1 / ( {2}^{g} ) ) \qquad Equation 501$$

where “g” is the generation number assigned to the airway in which the mucus plug is located. The mucus plug score is computed as the sum total of all such contributions in the lungs (or in a given portion of the lungs, or lobes of the lungs, etc.). Thus, a mucus plug having a higher generation number (e.g., located in an airway branch further downstream from the trachea, such as a 6^{th} or 7^{th} generation airway branch) will contribute less to the mucus plug score than a mucus plug having a lower generation number (e.g., located in an airway branch more upstream or closer to the trachea, such as a 2^{nd} or 3^{rd} generation airway branch).

FIG. 5 also shows equation 502, which is another alternative computation of a mucus plug score. In equation 502, each mucus plug contributes to the mucus plug score an amount equal to the inverse of the generation number, “g,” associated with or assigned to the given mucus plug, as follows:

$$mucus plug score = = \sum ( 1 / g ) \qquad Equation 502$$

Equation 503 in FIG. 5 provides yet another exemplary alternative way of computing a mucus plug score in which each mucus plug contributes to the mucus plug score an amount determined by a weighting factor or coefficient, “cg,” where the coefficients become somewhat smaller for each successive airway generation, for example. In some embodiments, the coefficients, “Cg,” may be chosen based on empirical data or results, or may be chosen in an attempt to generate mucus plug score values that may help characterize the risk presented by the extent of the mucus plugging associated with such resulting scores. For example, the coefficients may be chosen so that the resulting computed mucus plug score values tend to fall into ranges corresponding to risk classifications or categories, or based on clinical relevance to a healthcare professional.

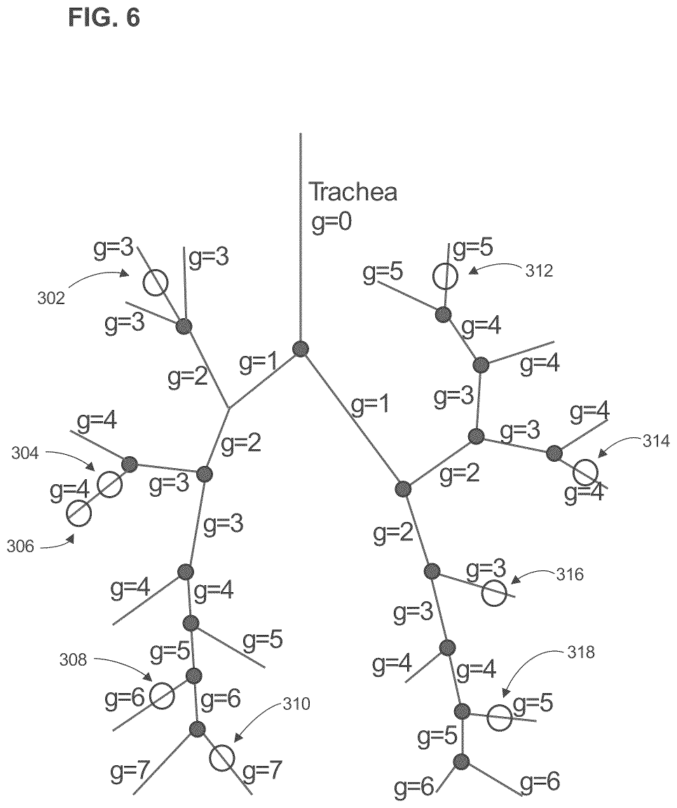

FIG. 6 is an exemplary airway branching structure of the lungs in which mucus plugs have been identified in nine locations, which are indicated with small circular dots and reference numerals ranging from 302 through 318 as shown in FIG. 6. In some embodiments, the presence of multiple mucus plugs in a given airway branch may count the same as a single plug for the purpose of computing a mucus plug score; that is, if one assumes that the presence of more than one plug in a given airway branch does not further worsen the condition for a patient, then the mucus plug score should account for this by not “over-counting” them. For example, mucus plugs 304 and 306 are shown in the same segmental airway branch in FIG. 6 and will be counted as a single plug in the exemplary mucus plug score computation that follows for the lungs shown in FIG. 6. As depicted in FIG. 6, mucus plugs are identified and assigned to corresponding airway generations as follows:

| | |
|---|---|
| Gen. 3: 302, 316 | 2 mucus plugs |
| Gen. 4: 304/306, 314 | 2 mucus plugs |
| Gen. 5: 312, 318 | 2 mucus plugs |
| Gen. 6: 308 | 1 mucus plug |
| Gen. 7: 310 | 1 mucus plug |

For the exemplary airway branching structure of FIG. 6, a mucus plug score may be computed using any of the equations 501, 502, or 503 described above with respect to FIG. 5. Using equation 501, for example, provides:

$$\begin{matrix}MP {S}_{501} = 2 * ( 1 / {2}^{3} ) + 2 * ( 1 / {2}^{4} ) + 2 * ( 1 / {2}^{5} ) + 1 * ( 1 / {2}^{6} ) + 1 * ( 1 / {2}^{7} ) \\ = 2 ( 0. 125 ) + 2 ( 0.0625 ) = 2 ( 0.03125 ) + 1 ( 0.015625 ) + 1 ( 0.0078125 ) \\ = 0.46 09375\end{matrix}$$

Alternatively, using equation 502, for example, may provide:

$$\begin{matrix}MP {S}_{502} = 2 * ( 1 / 3 ) + 2 * ( 1 / 4 ) + 2 * ( 1 / 5 ) + 1 * ( 1 / 6 ) + 1 * ( 1 / 7 ) \\ = 1.87 619\end{matrix}$$

It should be noted that, in some cases, the calculated mucus plug score (MPS) described above may be further modified and/or aggregated according to various selected portions of the lungs (e.g., aggregated by whole lung, right/left lung, lobe, and/or sub-lobe). Additionally, in some cases, grouping/aggregating/parsing of the mucus plug scores may be performed according to airway generation level. For example, it may be desirable to report a mucus plug score for mucus plugs occurring at each generation level.

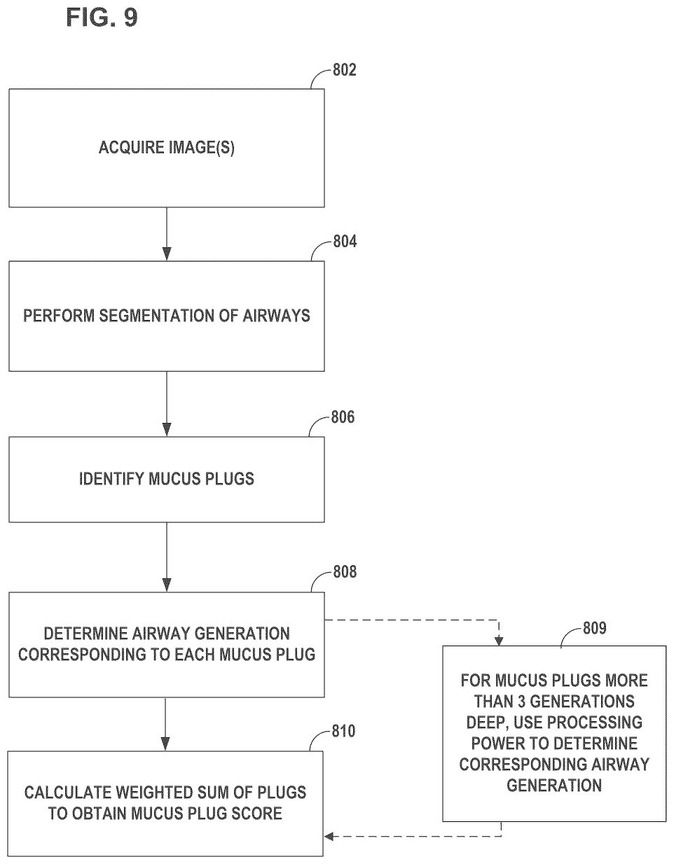

FIG. 9 is a flow diagram showing exemplary steps of an alternative method for assessing mucus plugs in a lung of a patient by determining a mucus plug score according to some embodiments of this disclosure. As can be seen, the method shown in FIG. 9 includes the same method steps depicted in the flow diagram of FIG. 3. For example, steps 802, 804, 806, 808 and 810 of FIG. 9 are identical to steps 1, 2, 3, 4 and 5 of FIG. 3. However, the method of FIG. 9 adds an additional optional step 809 which may or may not be performed following step 808. Optional step 809 provides that, for mucus plugs that are determined to be in an airway generation that is deeper than the third generation (e.g., a 4^{th} generation or higher airway), processing power is used to determine the airway generation corresponding to the mucus plug in question. A possible advantage of this technique is that it may conserve computer processing power (and processing time, etc.). In other words, if no mucus plugs are determined to be in a generation greater than the third generation, results may be obtained much more quickly. It is also possible that the optional step 809 could be skipped (e.g., at the option of a user) if, for example, the additional contribution to the mucus plug score is estimated to be very small. This may occur, for example, where there are several mucus plugs in second or third generation airways, and only a single additional mucus plug in an eighth-generation airway. The additional contribution to the total mucus plug score of the mucus plug in the eighth generation airway would likely be very small and unlikely to change the mucus plug score much; choosing to skip step 809 in such a case may be deemed beneficial from the perspective of conserving computer processing resources and/or time.

It should be noted that, in some cases, the calculated mucus plug score (MPS) described above may be further modified and/or aggregated according to various selected portions of the lungs (e.g., aggregated by whole lung, right/left lung, lobe, and/or sub-lobe). Additionally, in some cases, grouping/aggregating/parsing of the mucus plug scores may be performed according to airway generation level.

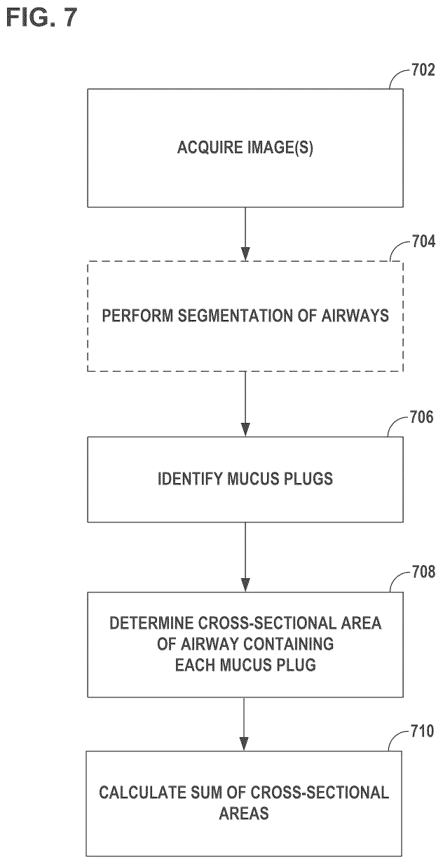

FIG. 7 is a flow diagram showing exemplary steps of a method of assessing mucus plugs in a lung of a patient according to certain embodiments of this disclosure. At step 702, images of the lung (or lungs, or a portion of a lung) are acquired. For example, radiological images or imaging data of a patient's lungs are transmitted to a pulmonary imaging system. The radiological images (e.g., volumetric radiological images) or imaging data may include CT scanned images or MRI scans, for example, from which a series of two-dimensional planar images can be produced in multiple planes, for example.

At step 704, segmentation of the airways may optionally be performed according to the method shown in FIG. 7. Airway segmentation, if performed at all in the method of FIG. 7, need not be done prior to identifying mucus plugs, and may be performed before, after, or in conjunction with performance of any other steps of this method as deemed appropriate. For example, the lungs, airways, and/or blood vessels may be segmented using the image data acquired in step 702. In some embodiments, a method may include processing the received volumetric pulmonary scan data to identify one or more anatomical structures within the volumetric pulmonary scan data. The methods of performing lung, airway and vessel segmentation from the volumetric images or imaging data may be those employed by the Pulmonary Workstation of VIDA Diagnostics, Inc. (Coralville, Iowa) as described hereinabove. Segmentation of the lungs, airways, and vessels results in identification of the lungs, airways, and vessels as distinct from the surrounding tissues and of separation of the lungs, airways, and vessels into smaller distinct portions which may be individually identified in accordance with standard pulmonary anatomy.

With continued reference to FIG. 7, step 706 involves identifying mucus plugs in the images of steps 702 and/or 704. Similar to the earlier-described method of FIG. 3, the identification of mucus plugs in the method of FIG. 7 may be performed automatically (e.g., via a neural network with deep learning trained to identify mucus plug candidates), manually (e.g., by experts or non-experts), or by using a combination of both.

In step 708, for each mucus plug identified in step 706, the cross-sectional area of the affected airway (e.g., the cross-sectional area of the airway at the point of blockage by the mucus plug) is determined corresponding to the location of each mucus plug in the airways of the lungs. With reference to the optional step 704 noted above (e.g., segmentation of airways), the cross-sectional area of the plugged area may be determined once the mucus plug is identified and located, and segmentation may be performed only for the airway or airways involved, according to some embodiments.

At step 710, a mucus plug score (or mucus plug severity score) is determined and/or calculated. The mucus plug score according to this embodiment may comprise a sum total of the cross-sectional areas of all mucus plugs identified. An example calculation may illustrate the computation of a mucus plug score according to such an embodiment. In an exemplary scan, 3 mucus plugs are identified having cross-sectional areas as follows:
1 mucus plug having a cross-sectional area of 0.3 cm²;
1 mucus plug having a cross-sectional area of 0.15 cm²; and
1 mucus plug having a cross-sectional area of 0.075 cm².

The Mucus Plug Score (“MPS”) for the above lungs may be determined according to the above-described method as:

$$MPS = ( 1 ) x ( 0.3 {cm}^{2} ) + ( 1 ) x ( 0.15 {cm}^{2} ) + ( 1 ) x ( 0.0 \; 75 {cm}^{2} ) = 0.5 \; 25 {cm}^{2}$$

It is worth noting that, in some cases, a mucus plug may be identified in an airway branch downstream of another mucus plug. In such cases, it may be desirable to not include downstream mucus plugs in the computation of a Mucus Plug Score using this method, for example.

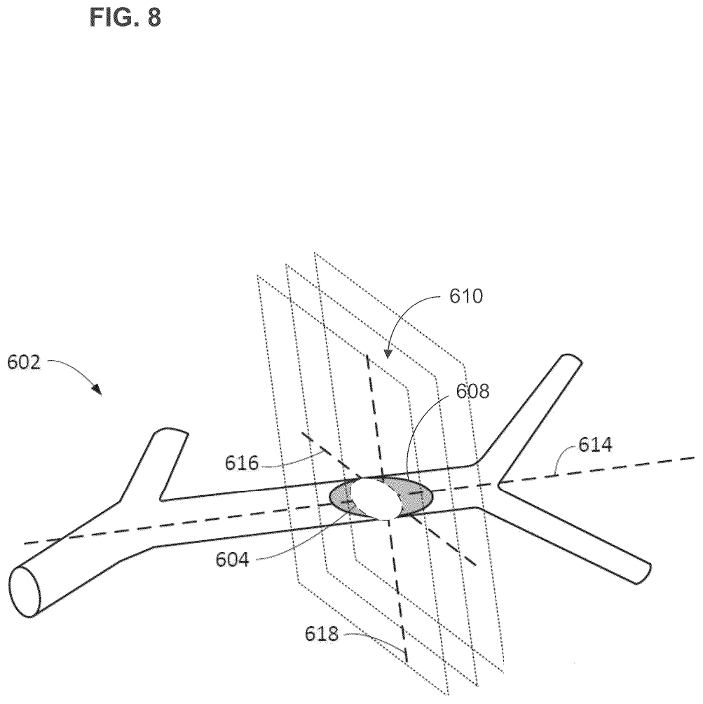

FIG. 8 is a schematic perspective image of an airway 602 of a portion of a lung. As shown, the airway 602 is being blocked by a mucus plug 610 obstructing the airway 602. The airway 602 may have a generally longitudinal axis 614 associated with it, and a corresponding pair of orthogonal or transverse axes, 616 and 618, extending generally horizontally and vertically, respectively, as shown in FIG. 8. Mucus plug 610 may have an overall three-dimensional shape, indicated generally by the shaded outer oval portion 608 in FIG. 8. Mucus plug 610 may also have an associated cross-sectional area 604 indicated by the oval dashed line 604 (shown with no shading). Cross-sectional area 604 is the obstructed area of the airway 602 and may lie in a plane defined by the pair of transverse axes 616 and 618, for example.

Referring again to FIG. 7, steps 708 and 710 involve determining the cross-sectional area of each mucus plug identified (e.g., cross-sectional area 604 of plug 610 in FIG. 8) and calculating a total sum of all such cross-sectional areas. The sum total of all such cross-sectional areas may be referred to as the total obstructed area, according to some embodiments. In some cases, the cross-sectional area determined may be aggregated according to various portions of the lungs (e.g., aggregated by whole lung, right/left lung, lobe, and/or sub-lobe). Additionally, in some cases, grouping/aggregating/parsing of the mucus plug scores (or of any related measures) may be performed according to airway generation level. For example, it may be desirable to report certain measures such as total obstructed cross-sectional area for mucus plugs occurring at each generation level.

In some embodiments, it may be desirable to calculate a “normalized” mucus plug score. A normalized score may, for example, allow for a comparison of mucus plug scores among a population of patients. (By contrast, an “un-normalized” score may be useful for trend analysis for a particular patient, but may not lend itself to comparisons to other patients.) A normalized score or normalized characterization of mucus plugging may be obtained by taking the raw or un-normalized score and dividing it by a patient-specific parameter. One example of normalizing the score would be to take the raw, mucus plug score of total obstructed area (e.g., obtained as generally described above with respect to FIG. 7), and dividing by a patient-specific parameter such as: (a) the total lung volume for the particular patient; (b) the total cross-sectional area of the segmental airways for the given patient; (c) the patient's weight; or (d) any other parameter that adjusts for differences in size. Referring again to the optional step 704 of FIG. 7 (segmentation of airways), it may be useful and/or desirable to perform segmentation of the airways to the extent needed to calculate a normalized mucus plug score (e.g., calculated by dividing the total obstructed area by the total cross-sectional area of the segmental airways), for example. Normalization may also (or alternatively) include adjusting the mucus plug score based on patient-specific factors such as age or gender so that the resulting numerical score may be compared to the same set of risk criteria. In other words, a normalized mucus plug score of greater than 1.5, for example, might put patients into a classification that requires further testing and/or indicates them for treatment, regardless of their size, age, gender, etc.

In some embodiments, a method of providing a mucus plug score may comprise determining a total plug mass of the mucus plugs identified. The total plug mass may be determined as the product of the volume and the density of each of the mucus plugs, summed together for all the mucus plugs in a relevant portion of the lungs. In some cases, the plug mass may be aggregated according to various portions of the lungs (e.g., aggregated by whole lung, right/left lung, lobe, and/or sub-lobe). Additionally, in some cases, grouping/aggregating/parsing of the mucus plug scores (or of any related measures) may be performed according to airway generation level. For example, it may be desirable to report certain measures such as total plug mass and/or total plug volume for mucus plugs occurring at each generation level.

In some embodiments, a method of providing a mucus plug score may be based on plug distribution. For example, the spatial density of the mucus plugs (e.g., number of plugs per cubic centimeter of lung) may be provided according to some embodiments. A mapping of the spatial density of the mucus plugs across the right and left lungs may thereby be provided. In some cases, the spatial density may be aggregated according to various portions of the lungs (e.g., aggregated by whole lung, right/left lung, lobe, and/or sub-lobe).

In some embodiments, a method of providing a mucus plug score may comprise determining a total volume of the “air-filled” or open airways downstream of the mucus plugs identified (e.g., excluding any mucus). Measuring such a volume could help determine how much airway could potentially be recovered if the obstructing mucus plugs were successfully cleared by administering a therapy, for example. The total volume of the downstream airways may be determined using volumetric image data to calculate the associated volumes of the affected airways. In some cases, the downstream airway volume may be aggregated according to various portions of the lungs (e.g., aggregated by whole lung, right/left lung, lobe, and/or sub-lobe). Additionally, in some cases, grouping/aggregating/parsing of the mucus plug scores (or of any of the related measures) may be performed according to airway generation level. For example, it may be desirable to report certain measures such as downstream airway volume for mucus plugs occurring at each generation level.

Additionally, in conjunction with any of the above-described methods, it may be useful and/or desirable to assess or measure changes over time by co-registering CT scans at two different timepoints and matching up mucus plugs that exist in both scans, for example.

In some embodiments, it may be desirable to characterize mucus plugging in the airways of the lungs of a patient by calculating a mucus plug score that reflects the total number of airway branches that are occluded and/or obstructed by one or more (e.g., at least one) mucus plugs. In such embodiments, the resulting mucus plug score (or mucus plugging score) will be a number (e.g., an integer whole number value) obtained by counting the number of airway branches that are occluded/obstructed by one or more mucus plugs. In some such embodiments, a single mucus plug could be counted multiple times. For example, a single mucus plug that occluded an airway branch upstream of a bifurcation, and extended downstream into two “child” branches (or sub-branches) at the bifurcation, this scenario would be counted as THREE airway branches obstructed as part of determining the total score according to this embodiment of a method of determining a mucus plug score. Conversely, one of those same sub-branches may also have additional downstream mucus plug obstructions in them, but those mucus plugs would not further add to the total mucus plug score according to this embodiment.

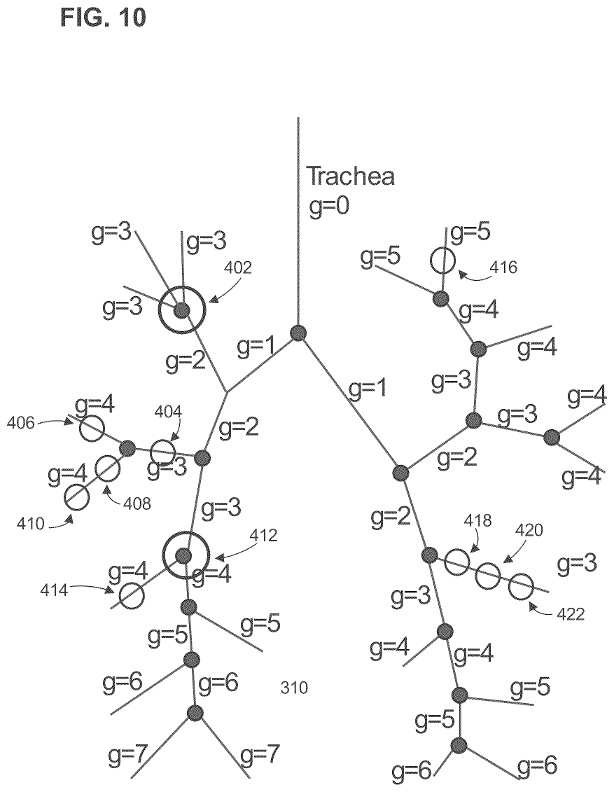

FIG. 10 is a schematic diagram showing an exemplary airway branch structure with mucus plug locations indicated to illustrate calculation of a mucus plug score according to the above-mentioned embodiment. As shown in FIG. 10, a number of mucus plugs have been illustrated (indicated with open circular areas) and identified with reference numerals ranging from 402 through 422 as shown. As discussed above, in this embodiment, the presence of multiple mucus plugs in a given airway branch may count the same as a single plug in the airway branch for the purpose of computing a mucus plug score, while a single mucus plug that extends from one airway branch into one or more downstream airway branches may be counted multiple times (e.g., once for each airway branch it obstructs).

For example, mucus plugs 402 is shown extending from a 2^{nd} generation branch into three 3^{rd} generation airway branches. This particular mucus plug 402 would contribute “four” to the count of obstructed airway branches according to this embodiment. Moving next to the branch structure including mucus plugs 404, 406, 408, and 410, these four mucus plugs would contribute “three” to the count of obstructed airway branches according to this embodiment, since plugs 408 and 410 reside in the same airway branch. Moving next to the branch structure including mucus plugs 412 and 414, these two mucus plugs would contribute “three” to the count of obstructed airway branches according to this embodiment, since plug 412 extends downstream from one airway branch into two sub-branches, and since 414 is not counted due to being in one of those same sub-branches. Moving next to the branch structure including mucus plug 416, this mucus plug would contribute “one” to the count of obstructed airway branches according to this embodiment. And finally, moving to the branch structure including mucus plugs 418, 420, and 422, these three mucus plugs would contribute “one” to the count of obstructed airway branches according to this embodiment, since all three plugs 418, 420, and 422, reside in the same airway branch.

Summing the total airway branches occluded or obstructed by one or more mucus plugs in the example illustrated in FIG. 10 yields a mucus plug score of 12 (equals 4+3+3+1+1).

A potential benefit of employing the above-described occluded airway branch count score is that the step of identifying and/or defining the airway branches in an airway tree is an established or understood process. One prior technique employed was to count the total number of mucus plugs were present in the airway branch structure. However, it can be difficult to determine whether and where one mucus plug ends and another begins. For example, mucus plugs sometimes have small air pockets in them that can blur the distinction between whether there is one contiguous mucus plug present versus two distinct mucus plugs. The above-described method of this disclosure overcomes this difficulty by instead counting each plugged airway branch as the individual unit to be counted. It should be noted that, in some scenarios, it is possible for a number of small mucus plugs to occur in close proximity to one another, and then grow larger and become a single mucus plug. Utilizing a straight counting technique to score the mucus plugging severity might seem to indicate that the mucus burden is getting lower because the plug count has been reduced. However, that is not the case in such a scenario. This type of anomaly is avoided by counting the number of plugged airway branches, which would stay the same in the scenario just described. Similarly, if a large mucus plug begins to break up into several disconnected smaller plugs, using a straight plug count might seem to indicate that the patient is getting worse since the mucus plug count would be getting higher. To the contrary, the plug breaking up may actually be the beginning of clearance. The airway branch count would tend to stay steady in this scenario as well.

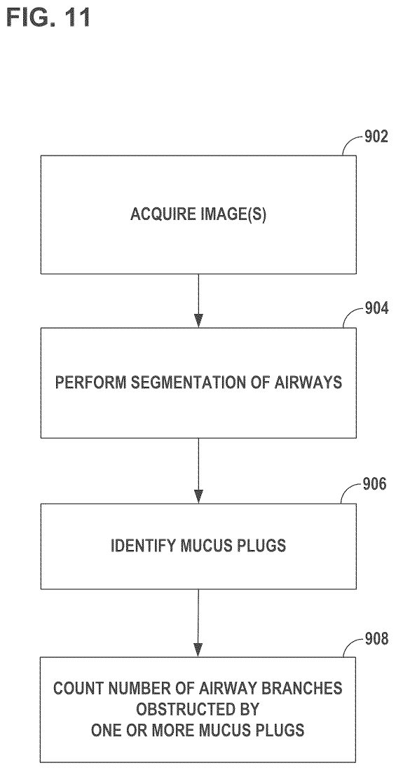

FIG. 11 is a flow diagram showing several steps of a method for determining a mucus plug score based on the above-described obstructed airway branch count method. For example, at step 902, image data corresponding to the airways of the patient's lung (or lungs, or a portion of a lung) is acquired. For example, radiological images or imaging data of a patient's lungs are transmitted to a pulmonary imaging system. The radiological images (e.g., volumetric radiological images) or imaging data may include CT scanned images or MRI scans, for example, from which a series of two-dimensional planar images can be produced in multiple planes, for example.

At step 904, segmentation of the airways is performed. For example, the lungs, airways, and/or blood vessels may be segmented by processing the image data acquired in step 902, for example, to identify a plurality of airway segments corresponding to a plurality of airway branches. In some embodiments, a method may include processing the received volumetric pulmonary scan data to identify one or more anatomical structures within the volumetric pulmonary scan data. The methods of performing lung, airway and vessel segmentation from the volumetric images or imaging data may be those employed by the Pulmonary Workstation of VIDA Diagnostics, Inc. (Coralville, Iowa) as described hereinabove. Segmentation of the lungs, airways, and vessels results in identification of the lungs, airways, and vessels as distinct from the surrounding tissues and of separation of the lungs, airways, and vessels into smaller distinct portions which may be individually identified in accordance with standard pulmonary anatomy.

With continued reference to FIG. 11, step 906 involves identifying one or more mucus plugs in the image data (e.g., in the airway branches provided by the images obtained in steps 902 and/or 904). Similar to the earlier-described methods of FIGS. 3 and 7, the identification of mucus plugs in the method of FIG. 11 may be performed automatically (e.g., via a neural network with deep learning trained to identify mucus plug candidates), manually (e.g., by experts or non-experts), or by using a combination of both.

In step 908, a numerical score is calculated by counting the airway branches that are occluded or obstructed by one or more of the mucus plugs identified in step 906. In some embodiments, a single mucus plug may count more than once (e.g., if it extends into more than one airway branch, such as at a bifurcation). Similarly, multiple mucus plugs located in a single airway branch will only count as one obstructed airway branch according to some embodiments.

Thus, a mucus plug score (or mucus plug severity score) is determined and/or calculated according to some embodiments as the total count of airway branches that are occluded or obstructed by one or more mucus plugs. In some embodiments, calculating a mucus plug score may further comprise using the numerical score to characterize the mucus plugging as being in a first risk category based on the numerical score being greater than a threshold. In some embodiments, calculating a mucus plug score may further comprise performing the calculation of the numerical score a second time and comparing a second calculated numerical score to a first calculated numerical score for a given patient. In some embodiments, multiple mucus plugs in a given airway branch may count as a single obstructed airway branch in performing the total count of the airway branches that are obstructed by one or more mucus plugs. In some embodiments, a single mucus plug that extends from a given airway branch into one or more downstream airway branches may count as two or more obstructed airway branches in performing the total count of the airway branches that are obstructed by one or more mucus plugs.

Of course, other comparable scores may be defined as needed and/or as technology evolves in the future, and such methods are contemplated and deemed to be within the scope of this disclosure.

In the foregoing detailed description, inventive concepts have been described with reference to various illustrative embodiments. However, it may be appreciated that various modifications and changes can be made without departing from the scope of the invention.

What is claimed is:

1. A method of characterizing mucus plugging in airways of lungs of a patient, the method comprising:
acquiring image data corresponding to the airways of the patient's lungs;
processing the image data to identify a plurality of airway segments;
identifying one or more mucus plugs in the image data;
for each of the one or more mucus plugs identified, determining an airway generation corresponding to a location of each mucus plug; and
calculating a numerical score to characterize the mucus plugging, the numerical score comprising a weighted sum total of the mucus plugs based on the corresponding airway generation of each of the one or more mucus plugs.

2. The method of claim 1, further comprising:
using the numerical score to characterize the mucus plugging as being in a first risk category based on the numerical score being greater than a threshold.

3. The method of claim 1, further comprising:
performing the method of characterizing mucus plugging in the airways of the lungs of the patient a first time to calculate a first numerical score;
performing the method of characterizing mucus plugging in the airways of the lungs of the patient a second time to calculate a second numerical score; and
comparing the second numerical score to the first numerical score for the patient.

4. A method of characterizing mucus plugging in airways of lungs of a patient, the method comprising:
acquiring image data corresponding to the airways of the patient's lungs;
identifying one or more mucus plugs in the image data;
for each of the one or more mucus plugs identified, determining a cross-sectional area of an airway segment corresponding to a location of each mucus plug; and
calculating a numerical score to characterize the mucus plugging, the numerical score comprising a sum total of the cross-sectional areas of all of the mucus plugs identified.

5. The method of claim 4, further comprising:
using the numerical score to characterize the mucus plugging as being in a first risk category based on the numerical score being greater than a threshold.

6. The method of claim 4, further comprising:
determining a normalized characterization of mucus plugging by dividing the numerical score by a patient-specific parameter.

7. The method of claim 6, wherein the patient-specific parameter is total lung volume for the given patient.

8. The method of claim 6, further comprising:
processing the image data to identify a plurality of airway segments,
wherein the patient-specific parameter is a total cross-sectional area of the airway segments for the given patient.

9. The method of claim 4, further comprising:
performing the method of characterizing mucus plugging in the airways of the lungs of the patient a first time to calculate a first numerical score;
performing the method of characterizing mucus plugging in the airways of the lungs of the patient a second time to calculate a second numerical score; and
comparing the second numerical score to the first numerical score for the patient.

10. A method of characterizing mucus plugging in airways of lungs of a patient, the method comprising:
acquiring volumetric image data corresponding to the airways of the patient's lungs;
identifying one or more mucus plugs in the image data;
for each of the one or more mucus plugs identified, determining a volume of open airway downstream of each mucus plug; and
calculating a numerical score to characterize the mucus plugging, the numerical score comprising a sum total of the volumes of open airway downstream of all of the one or more mucus plugs.

11. The method of claim 10, further comprising:
using the numerical score to characterize the mucus plugging as being in a first risk category based on the numerical score being greater than a threshold.

12. The method of claim 10, further comprising:
determining a normalized characterization of mucus plugging by dividing the numerical score by a patient-specific parameter.

13. The method of claim 12, wherein the patient-specific parameter is total lung volume for the given patient.

14. The method of claim 12, further comprising:
processing the image data to identify a plurality of airway segments,
wherein the patient-specific parameter is a total cross-sectional area of the airway segments for the given patient.

15. The method of claim 10, further comprising:
performing the method of characterizing mucus plugging in the airways of the lungs of the patient a first time to calculate a first numerical score;
performing the method of characterizing mucus plugging in the airways of the lungs of the patient a second time to calculate a second numerical score; and
comparing the second numerical score to the first numerical score for the patient.

16. A method of characterizing mucus plugging in airways of lungs of a patient, the method comprising:
acquiring image data corresponding to the airways of the patient's lungs;
processing the image data to identify a plurality of airway segments corresponding to a plurality of airway branches;
identifying one or more mucus plugs in the image data; and
calculating a numerical score to characterize the mucus plugging, the numerical score comprising a total count of the airway branches that are obstructed by one or more mucus plugs.

17. The method of claim 16, further comprising:
using the numerical score to characterize the mucus plugging as being in a first risk category based on the numerical score being greater than a threshold.

18. The method of claim 16, further comprising:
performing the method of characterizing mucus plugging in the airways of the lungs of the patient a first time to calculate a first numerical score;
performing the method of characterizing mucus plugging in the airways of the lungs of the patient a second time to calculate a second numerical score; and
comparing the second numerical score to the first numerical score for the patient.

19. The method of claim 16, wherein multiple mucus plugs in a given airway branch counts as a single obstructed airway branch in the total count of the airway branches that are obstructed by one or more mucus plugs.

20. The method of claim 16, wherein a single mucus plug that extends from a given airway branch into one or more downstream airway branches may count as two or more obstructed airway branches in the total count of the airway branches that are obstructed by one or more mucus plugs.
